(19) United States
(12) Patent Application Publication
(10) Pub. No.: US 2026/0221290 A1
(43) Pub. Date: Jul. 30, 2026

(54) METHOD FOR CONSTRUCTING A MEDICAL MODEL, SYSTEM AND STORAGE MEDIUM THEREOF

(71) Applicant: Taipei Medical University, Taipei City (TW)

(72) Inventors: Yung-Chun Chang, Taipei City (TW); Ting-Yun Huang, Taipei City (TW)

(21) Appl. No.: 19/037,346

(22) Filed: Jan. 27, 2025

Related U.S. Application Data
(60) Provisional application No. 63/603,651, filed on Nov. 29, 2023.

(51) Int. Cl. G16H 50/50; G06F 40/166; G06F 40/232; G06F 40/279; G06F 40/58; G16H 10/60; G16H 40/20; G16H 50/20

(52) U.S. Cl. CPC G16H 50/50; G06F 40/166; G06F 40/232; G06F 40/279; G06F 40/58; G16H 10/60; G16H 40/20; G16H 50/20

(57) ABSTRACT

A method for constructing a medical model and a medical model are disclosed. The medical model is configured to predict a diagnosis result based on information of target patients, and the method comprising: acquiring a first data set of a medical institution, wherein the first data set comprises a first structured data and a first unstructured data, and the first structured data and the first unstructured data are both in text form; preprocessing the first data set into a second data set, the second data set comprises a second structured data and a second unstructured data; obtaining a first type of data based on the second structured data, and processing the second unstructured data by a bidirectional long short-term memory network to obtain a second type of data, and extracting language features from the second unstructured data to obtain a third type of data; fusing the first type of data, the second type of data, and the third type of data, and processing the fused result using a softmax to obtain the diagnosis result. The medical model can predict patients' outcomes to identify potential critical patients.

### CROSS-REFERENCE TO RELATED APPLICATIONS

This application claims priority of Provisional Application No. 63/603,651, filed on Nov. 29, 2023, the content of which is incorporated herein in its entirety by reference.

### FIELD OF THE INVENTION

The present application relates to the field of data processing technology, and specifically relates to a method for constructing a medical model and a medical model.

### BACKGROUND OF THE INVENTION

In some scenarios, when a patient enters a hospital for treatment, medical staff (i.g., nurse)-led triage is first used to make a preliminary assessment of the patient's condition, and then the patient is sent to a doctor for further examination. Due to limited medical resources, if a patient is marked as a non-critical patient by a nurse, he may not be able to get a doctor's diagnosis in time; relatively, patients marked as critical patients by nurses are more likely to get a doctor's diagnosis first.

However, medical staff-led triage may sometimes fail to identify potential critical patients accurately. For example, when a potential critical patient is marked as a non-critical patient by a nurse, he may become more ill or even die due to a long wait. Therefore, there is an urgent need for some effective technical means to identify potential critical patients marked as non-critical patients in the nurse-led triage process to improve the patient's medical experience.

Around the world, emergency departments (EDs) frequently experience overcrowding, which can delay critical medical care. Given that the prognosis for critically ill patients is highly sensitive to timely interventions, early identification of patients in deteriorating conditions is essential. Identifying patients who need hospitalization and the appropriate medical care has always posed challenges for emergency physicians. Clinical judgment, which depends on laboratory results and physicians' prior experiences, can be influenced by individual factors, potentially increasing morbidity and mortality. Among non-hospitalized patients, 2-5% revisit the emergency department (ED) within 72 hours. Of these, 19%-30% are admitted to the hospital, and one-third experience adverse events. Unnecessary tests and treatments during the second ED visit increase costs and prolong stays.

Therefore, creating a clinical decision support system with a medical model to predict the disposition of emergency patients can help minimize human error. This system can also reduce length of stay (LOS), enhance the quality of medical care, and optimize the allocation of medical resources.

### SUMMARY OF THE INVENTION

In view of the foregoing, the present invention discloses a method for constructing a medical model, a medical system having such a medical model, and a storage medium containing such a medical model, and the medical model is used to predict a patient's condition or a diagnosis based on patient information, to identify potentially critically ill patients, and to reduce the probability of deterioration of a potentially critically ill patient's condition due to a long waiting period. Probability of deterioration due to long waiting times for potentially critically ill patients.

In the first aspect, the present application discloses an embodiment of a method for constructing a medical model. The medical model is configured to predict a diagnosis result based on a subject's information. The method includes the following steps: (A) acquiring a first data set of a medical institution, wherein the first data set comprises a first structured data and a first unstructured data; (B) preprocessing the first data set into a second data set, wherein the second data set comprises a second structured data and a second unstructured data; (C) obtaining a first type of data based on the second structured data, and processing the second unstructured data by a bidirectional long short-term memory network to obtain a second type of data, and extracting linguistic features from the second unstructured data to obtain a third type of data; and (D) fusing the first type of data, the second type of data, and the third type of data to get a fused result, wherein the fused result is processed using a softmax function to obtain the diagnosis result.

In some embodiments, Step (B) further includes (B1) grouping the first structured data based on a preset index and normalizing the first structured data; and (B2) optimizing the first unstructured data so that the first unstructured data matches a rule of the medical model, wherein an optimization comprises at least one step of data cleaning, text translation, or text correction. The medical model processes the first unstructured data using a natural language processing algorithm, and the natural language processing algorithm is designed to process monolingual information.

In some embodiments, a reference quantity for grouping comprises age, mean arterial pressure, or body temperature.

In some embodiments, Step (B1) includes performing a one-hot encoding on a grouped first structured data and normalizing a respiratory rate, a pulse rate, and a blood oxygen saturation in the first structured data by a min-max normalization algorithm.

In some embodiments, the data cleaning includes converting texts in the first unstructured data in abbreviated form into terms in fully expanded form. In some preferable embodiment, the data cleaning includes retaining preset punctuation marks in the first unstructured data and deleting stop words in the first unstructured data and punctuation marks other than the preset punctuation marks, wherein the preset punctuation marks are commonly used punctuation marks in a medical field. In some preferable embodiments, the data cleaning includes retaining negative words in the first unstructured data.

In some embodiments, the preset punctuation marks include a slash, a plus sign, a minus sign, and a space character.

In some embodiments, the text translation includes translating non-English texts in the first unstructured data into English texts.

In some embodiments, the text correction includes correcting spelling errors in the first unstructured data.

In some embodiments, Step (A) further includes filtering the first data set to exclude data of non-target patients in the first data set, wherein the data of non-target patients comprise data of non-adults, data of people who suffer an out-of-hospital cardiac arrest, data of people who discharge against medical advice, data of people who leave without notice, data of people who withdraw registrations, data of people who are transferred to other institutions, and data of people with incomplete data.

In some embodiments, the diagnosis result comprises a critical outcome or a non-critical outcome, wherein the critical outcome comprises an in-hospital cardiac arrest or an admission to an intensive care unit, and the non-critical outcome comprises a discharge or an admission to a ward.

In some embodiments, the second structured data includes vital signs and demographic data, and the second unstructured data comprises a chief complaint, a present illness, and a medical history. The second type of data includes a first sub-data and a second sub-data, and the third type of data comprises a third sub-data, a fourth sub-data, and a fifth sub-data. Further, the first sub-data is obtained by processing the chief complaint through the bidirectional long short-term memory network, the second sub-data is obtained by processing the present illness through the bidirectional long short-term memory network, the third sub-data is obtained by extracting linguistic features from the chief complaint, the fourth sub-data is obtained by extracting linguistic features from the medical history, and the fifth sub-data is obtained by extracting linguistic features from the present illness.

In some embodiments, the first sub-data is related to the vital signs and the demographic data.

In some embodiments, a dimension of the data related to the chief complaint and the medical history in the second and third types of data is between 100-500.

In some embodiments, the dimension of the first sub-data and the second sub-data are both 300.

In some embodiments, the dimensions of the third and fifth sub-data are both 200.

In some embodiments, the dimension of the fourth sub-data is 30.

In some embodiments, the dimension of the first type of data is 15.

In some embodiments, the dimension of the fused result is 64.

In some embodiments, the step of fusing the first type of data, the second type of data, and the third type of data to get a fused result include the following calculation formulas:

$$F = DF ⊕ VF ⊕ LF ⊕ LSF$$

$$\hat{y} = softmax ( {MF}^{T} + b )$$

Further, F is a result of the fusion, DF is a feature related to the demographic data in the first type of data, VF is a feature related to the vital signs in the first type of data, LF is the third type of data, LSF is the second type of data, ŷ is an approximate probability value, M is a parameter matrix of a connection layer, and b is a bias.

In some embodiments, the step of “extracting linguistic features from the second unstructured data to obtain a third type of data” further includes: when the second unstructured data contains keywords, extract the keywords from the second unstructured data, and obtain the third type of data based on the keywords and corresponding weights; and when the second unstructured data do not contain keywords, average values of all word vectors in the second unstructured data are calculated, and cosine similarities between the average values and all preset keyword vectors is calculated, and five preset keyword vectors with a closest distance are selected as the third type of data.

In some embodiments, a weight calculation formula is as follows:

$$LLR ( w , HR ) = 2 log [ \frac{{p ( w \vert HR )}^{k} {( 1 - p ( w \vert HR ) )}^{m} {p ( w \vert ¬ HR )}^{l} {( 1 - p ( w \vert ¬ HR ) )}^{n}}{{p ( w )}^{k + l} {( 1 - p ( w ) )}^{m + n}} ]$$

Further, HR is a high-risk patient, ¬ HR is a non-high-risk patient, w represents a keyword of high risk, k represents a quantity of clinical narratives of high-risk cases including w, l represents a quantity of clinical narratives including w but not high-risk cases, m represents a quantity of clinical narratives of high-risk cases without w, and n represents a quantity of clinical narratives of non-high-risk cases without w.

In some embodiments, the medical model is constructed using following parameters: a validation method is 10-fold cross-validation, a dropout is 0.35, training epochs are 40, a threshold is 0.5, a parameter of the early stopping strategy is 3, and a maximum length of each text is 350. The method further includes a synthetic minority class oversampling technique is used to balance a data distribution so that a proportion of data in each class is equal.

In the second aspect, the present disclosure further includes a medical system having a medical model, which is constructed using the method described in above, wherein the medical model is configured to predict a diagnosis result based on information of target patients.

In the third aspect, the present disclosure further includes a non-transitory computer-readable storage medium having computer-executable instructions stored thereon, which when executed by one or more processors, cause the one or more processors to perform operations having the above method for constructing a medical model to predict a diagnosis result based on a subject's information.

In summary, the method for constructing a medical model and the medical model disclosed in this application have at least the following beneficial effects:

(1) The medical model disclosed in this application can predict possible diagnostic results based on the medical history and vital signs of the patient routinely collected during triage. The predicted diagnostic results can assist doctors in making diagnoses and reduce the probability of misdiagnosis.

(2) Compared with the prior art that uses digital data for calculations, both structured data and unstructured data are used in the process of building the medical model. The data input into the medical model are all in text form, and these data are combined with the language processing module in the medical model to make the predicted diagnostic results more accurate.

(3) The area under the receiver operating characteristic curve (AUROC) of the medical model disclosed in this application is 0.874, and the area under the precision-recall curve (AUPRC) is 0.207, which are better than other related medical models in the prior art; and are better than the processing level of doctors in terms of sensitivity and accuracy.

### BRIEF DESCRIPTION OF DRAWINGS

One or more specific embodiments are shown in the accompanying drawings by way of example and not by way of limitation. The accompanying drawings are not to scale unless otherwise disclosed. Those with ordinary knowledge of the art should understand the present disclosure in light of the following detailed description of preferred specific embodiments and with reference to the accompanying drawings.

The above features, technical characteristics, advantages, and their realizations of the present disclosure will be further described below in a clear and easy-to-understand manner in conjunction with the illustrations of the preferred specific embodiments.

FIG. 1 shows a flow chart of a method for constructing a medical model provided in an embodiment of the present application.

FIG. 2 shows a schematic diagram of a medical model provided in an embodiment of the present application.

FIG. 3 shows a method for processing clinical narratives without any keywords in an embodiment of the present application.

FIG. 4 shows a schematic diagram of a medical model provided in an embodiment of the present application.

### DETAILED DESCRIPTION OF THE INVENTION

The following descriptions in the present disclosure are primarily intended to illustrate the content of the disclosure and not to limit its scope. Therefore, specific details such as system architecture and techniques are provided to thoroughly explain relevant specific embodiments of this application. However, one person having ordinary skill in the art to which this invention pertains should be able to implement the essence of this disclosure without these specific details. In certain instances, detailed descriptions of well-known systems, devices, circuits, and methods are omitted to avoid unnecessary details that might obscure the description of the disclosure.

In order to simplify the drawings, only the parts related to the corresponding embodiments are schematically shown in each figure, and they do not represent the actual structure of the product. In addition, in order to simplify the drawings and facilitate understanding, only part of the components with the same structure or function in some figures are schematically drawn, and there may be more or fewer components with the same structure or function in reality.

It should be understood that when terms like “comprising,” “including,” or “includes” are used in the specification and claims of this disclosure, they indicate the presence of described features, integrals, steps, operations, elements, and/or components but do not exclude the presence or addition of one or more other features, integrals, steps, operations, elements, components, and/or collections.

For clarity in the drawings, only parts related to the disclosure are shown schematically, and they do not represent the actual structure as a product. Furthermore, for simplicity and ease of understanding, in some figures, parts with the same structure or function are schematically shown, or only one of them is labeled. Herein, “one” does not only mean “only one” but can also mean “more than one.”

In this application, unless otherwise clearly specified and limited, ordinal numbers, such as “first,” “second,” etc., are only used to distinguish and describe associated objects and cannot be understood as indicating or implying the relative importance or order between associated objects; in addition, they do not represent the number of associated objects. “Multiple” includes two or more, and other quantifiers are similar. “/” describes the relationship between associated objects, which indicates the “or” relationship between associated objects. “And/or” is used to describe the relationship between associated objects, which includes any combination relationship between associated objects, such as “a and/or b” includes: “alone a,” “alone b,” or “a and b.” “One or more” or “at least one” in multiple objects refers to any object or any combination of multiple objects, such as “one or more of a1, a2, a3” or “at least one of a1, a2, a3” includes: “alone a1”, “alone a2”, “alone a3”, “a1 and a2”, “a1 and a3”, “a2 and a3”, or “a1, a2 and a3”.

Due to the growing demand for medical care and the shortage of medical staff, more and more hospitals are facing the problem of shortage of medical resources. The shortage of medical resources is reflected in various aspects of hospitals, such as overloaded emergency rooms, shortage of intensive care unit (ICU) beds, long waiting times for surgery, shortage of drugs and equipment, etc. Among them, emergency department (ED) crowding is a universal phenomenon, and many studies have shown that ED crowding contributes to delays in critical care. Since the outcomes of many ED patients' medical treatment is time-sensitive, early identification of potential critical patients is essential in lowering morbidity and mortality. In addition, ED crowding affects the quality of medical care. Long ED stays not only negatively impact patient satisfaction but also increase medical costs. Therefore, early identification of critical patients will help reduce hospital stays and costs and improve the quality of medical care.

Nurse-led triage is the current international standard triage model. For example, in Taiwan, the Chinese version of the Canadian Emergency Department Triage and Acuity Scale (CTAS) is applied in daily practice. Patients are first categorized into five levels of urgency by a triage nurse based on vital signs and chief complaints before being reviewed by an emergency physician, who then records patient history (chief complaints, present illness, and medical history), conducts a physical examination, and orders serology and image tests. The five levels of urgency of CTAS are arranged in order of importance: resuscitation, emergency, urgent, less urgent, and non-urgent. Different treatment methods are used for different levels of urgency.

Due to limited medical resources, if a patient is marked as a non-critical patient by a nurse, he may not be able to get a doctor's diagnosis in time; in other words, patients marked as critical by nurses are more likely to get a doctor's diagnosis first. For example, if the triage nurse determines a patient's condition to be less urgent or non-urgent, he will be marked as a non-critical patient; for another example, if a patient's condition is determined by the triage nurse to be resuscitation, emergency, or urgent, he will be marked as a critical patient. It should be noted that different hospitals may adopt different standards for the above classifications, and the above content is only for reference examples.

However, nurse-led triage may sometimes fail to identify potential critical patients accurately. For example, when a potential critical patient is marked as a non-critical patient by a nurse, his condition may worsen or even die due to a long wait. However, if the initial triage is performed directly by a professional doctor, it will lead to a shortage of medical resources. Therefore, the core concept of this application is that under the premise of maintaining the existing nurse-led triage, a medical model is designed using advanced technical means to predict the diagnosis results based on the information of the target patient. The diagnosis results predicted by the medical model can assist nurses or doctors in identifying potential critical patients, thereby reducing the probability of misdiagnosis, reducing the probability of potential critical patients' condition worsening due to long waiting, and improving the patient's medical experience.

The following is a description with reference to the accompanying drawings.

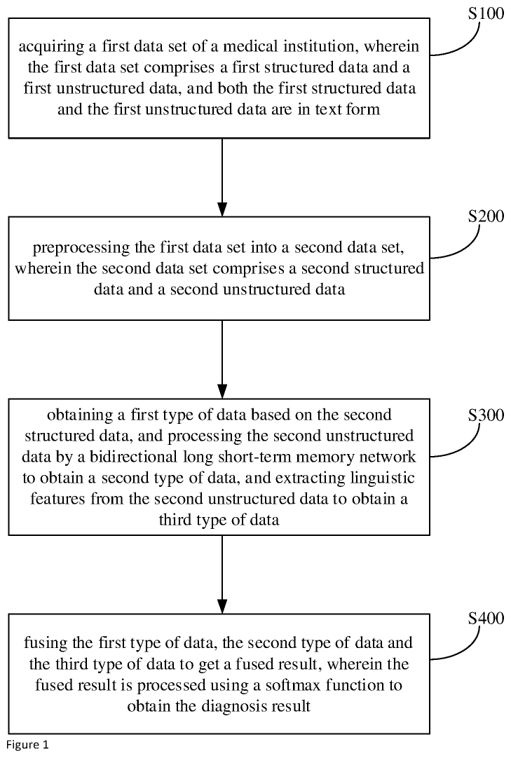

FIG. 1 shows a flow chart of a method for constructing a medical model provided in an embodiment of the present application. Referring to FIG. 1, the medical model is configured to predict a diagnosis result based on information from a target patient, and the method includes the following steps:

Sept S100: acquiring a first data set of a medical institution, wherein the first data set includes the first structured data and the first unstructured data, and both the first structured data and the first unstructured data are in text form. In other words, both the First Structured Data and the First Unstructured Data are either pre-transformed or directly into textual form before being collected.

Step S200: preprocessing the first data set into a second data set, wherein the second data set comprises a second structured data and a second unstructured data;

Step S300: obtaining a first type of data based on the second structured data, and processing the second unstructured data by a bidirectional long short-term memory network to obtain a second type of data, and extracting linguistic features from the second unstructured data to obtain a third type of data. In addition, the linguistic feature extraction is processed using Natural Language Processing (NLP).

Step S400: fusing the first type of data, the second type of data and the third type of data to get a fused result, wherein the fused result is processed using a softmax function to obtain the diagnosis result.

The method for constructing a medical model disclosed in the present application can be generally divided into acquiring data, preprocessing the data, classifying the preprocessed data and performing different operations respectively, fusing the data, and obtaining a predicted diagnostic result based on the fused result.

In order to improve the authenticity of the data and the reliability of the medical model, the data set used in this application comes from medical institutions. The medical institution may be, for example, Taipei Medical University-Shuang Ho Hospital, Ministry of Health and Welfare, and the data set may be the clinical data of the emergency department of the medical institution. Since the medical institution has approximately 95,000 emergency department data each year, the data set obtained from the medical institution has a greater reference significance. In other words, when constructing a medical model, this application has high requirements for the source of the data set: the data used in constructing the medical model are all real medical data of the patient.

For the sake of distinction, the data set from the medical institution is referred to as the first data set; and the first data set includes two types of data: the first structured data and the first unstructured data. The first structured data may include demographic data, such as age and gender; the way of arriving (to the hospital), such as ambulance, walk-in by himself/herself, walk-in accompanied by a third party, or transferred from other medical institutions; vital signs, such as body temperature, pulse, respiratory rate, blood oxygen saturation, and blood pressure. The first unstructured data may include chief complaint, present illness, and medical history. Whether it is the first structured data or the first unstructured data above, their expression form is text. Among them, the first unstructured data can also be called the patient's clinical narrative; the original expression form of this type of data may be a recording, and their expression form can be converted into text using a device with a text conversion function. For the first structured data, their original expression form may be in digital form; for example, for a patient with a body temperature of 39 degrees Celsius, the value one (V=1) can be used in the prior art to represent that the patient is in a fever state, and for a patient with a body temperature of 37 degrees Celsius, the value zero (V=0) can be used in the prior art to represent that the patient is not in a fever state. Different from the above processing methods of the prior art, in this application, for the above patients with a body temperature of 39 degrees Celsius, the text form such as “patient with fever” is directly used to express this information. That is to say, no matter what the original expression form of the first structured data and the first unstructured data is, before these data are used to build a medical model, their expression forms will first be converted into text form (if their original expression forms are not text forms). The reason for converting the above data into text form is that these data can be coordinated with the language processing module in the medical model. In the subsequent data processing process, the diagnosis result of the target patient will be predicted based on the similarity between each word in the text and the preset keywords. Therefore, converting the data belonging to the medical model into text form in advance can make the predicted diagnosis result more accurate.

In the process of constructing the medical model, the first data set will first undergo preprocessing so that errors or defects in the first structured data and the first unstructured data in the first data set are corrected. Preprocessing includes grouping, standardization, data cleaning, text translation, and text correction. The data obtained after preprocessing is called the second data set for the sake of distinction. The second data set includes the second structured data and the second unstructured data. The second structured data corresponds to the first structured data, and the second unstructured data corresponds to the first unstructured data. When building a medical model, preprocessing the data input into the model can improve the convergence speed of the model. For example, through operations such as standardization and normalization, the characteristic values of the data are distributed within a specific range, reducing the magnitude difference between different features, which helps the model find the optimal solution faster, thereby accelerating convergence. In addition, considering that some of the data in the first data set are quick doctors' records during consultations, different doctors have different recording habits; the quick records include abbreviations and spelling errors. Therefore, preprocessing can enable the model to learn better data, achieving better performance and prediction results.

After the preprocessing is completed, the second structured data and the second unstructured data in the second data set are processed separately. For the second unstructured data, the bidirectional long short-term memory network (BILSTM) and language feature extraction are used to obtain two different types of data, namely the second type of data and the third type of data. The first type of data is obtained based on the second structured data. The first type of data, the second type of data, and the third type of data are fused. The fused result is the characterization of the fusion distributed characteristics. Finally, the softmax function is used to process the fused result to obtain the predicted diagnosis result. The calculation formula is as follows:

$$F = DF ⊕ VF ⊕ LF ⊕ LSF ;$$

$$\hat{y} = softmax ( {MF}^{T} + b ) ;$$

Among them, DF is the demographic features, VFVF is the vital sign features, LF is the linguistic features (language features), LSF is the latent semantic features, ŷ is the approximate probability value, M is the parameter matrix of the connection layer (in the medical model), F is the representation of the fusion distribution features (namely the fused result), and b is the bias. The demographic features and vital sign features come from the first type of data above, the latent semantic features are the second type of data above, and the language features are the third type of data above. The above formula can get two approximate probability values, and the larger value is taken as the final result, which is the diagnosis result predicted by the medical model.

During the fusion process, the fused result is expressed in 64 dimensions. In this application, the experiment is carried out in the form of 2 exponentially from 32 (2 to the 5th power) to 128 (2 to the 7th power), and it is finally determined that when the dimension is 64, the best effect can be achieved: that is, the vector representation is simplified while retaining the key information.

Moreover, considering the convergence rate, the ReLU function is used as an activation function for nonlinear operation, and the dropout mechanism is used as a correction to prevent overfitting. The Adam optimizer is used since it can optimize the loss function of the network, which has been shown to be an effective and efficient backpropagation algorithm. This application employs the cross-entropy function as the loss function because it can reduce the risk of a gradient disappearance during the process of stochastic gradient descent, which means it is often better than the classification error rate or the mean square error.

The loss rate of the model can be calculated as follows.

$$Loss = - {\sum }_{i = 1}^{N} {y}_{i} \times log {\hat{y}}_{i} + ( 1 - {y}_{i} ) \times log ( 1 - {\hat{y}}_{i} )$$

Where N is the number of training samples, y is the sample's label, and ŷ is the output of the model.

In some embodiments of the present application, the diagnosis result includes a critical outcome or a non-critical outcome, wherein the critical outcome includes an in-hospital cardiac arrest or an admission to an intensive care unit, and the non-critical outcome includes a discharge or an admission to a ward.

The medical model constructed according to the above method can predict possible diagnostic results according to the patient's medical history and vital signs routinely collected during triage when in use. The predicted diagnostic results can assist doctors in diagnosis and reduce the probability of misdiagnosis. Compared with the prior art of using digital data for calculation, structured and unstructured data are used simultaneously in building the medical model. The data input into the medical model are expressed in text form, and these data are matched with the language processing module in the medical model, which can make the predicted diagnostic results more accurate.

In a specific embodiment of the present invention, the above medical model is constructed to assess whether the patient visiting an emergency clinic is truly in critical condition. After the medical model is built, its performance is tested. As Table 1 shows, its area under the receiver operating characteristic curve (AUROC) is 0.874, and its area under the precision-recall curve (AUPRC) is 0.207, which is better than other related medical models in the prior art, and it is better than the doctor's processing level in terms of sensitivity and accuracy (as Table 2 shows). Since the medical model of this application is based on the existing triage system (that is, the nurse-led triage mentioned above), it can identify potential critical patients, so it may also be used in the training of emergency doctors to cultivate the ability of doctors to make decisions under incomplete information and time pressure.

TABLE 1
Comparison of the Performance of Various Natural Language Processing Models

| Method | AUROC | P value | AUPRC | Sensitivity | Specificity | PPV | NPV |
|---|---|---|---|---|---|---|---|
| NB | 0.785 | <0.001 | 0.143 | 0.556 | 0.868 | 0.098 | 0.987 |
| RF | 0.789 | <0.001 | 0.134 | 0.115 | 0.990 | 0.224 | 0.977 |
| XGB | 0.783 | <0.001 | 0.084 | 0.193 | 0.961 | 0.113 | 0.979 |
| LR | 0.828 | <0.001 | 0.133 | 0.685 | 0.779 | 0.074 | 0.990 |
| MLP | 0.765 | <0.001 | 0.100 | 0.210 | 0.965 | 0.135 | 0.979 |
| TextCNN | 0.786 | <0.001 | 0.090 | 0.285 | 0.931 | 0.096 | 0.981 |
| BERT | 0.810 | <0.001 | 0.180 | 0.069 | 0.998 | 0.490 | 0.977 |
| BILSTM | 0.807 | <0.001 | 0.133 | 0.168 | 0.981 | 0.188 | 0.979 |
| BILSTM + | 0.844 | <0.001 | 0.157 | 0.452 | 0.937 | 0.156 | 0.985 |
| TR | | | | | | | |
| Present | 0.847 | — | 0.207 | 0.499 | 0.933 | 0.161 | 0.986 |
| Model | | | | | | | |

TABLE 2
Predictive performance of emergency physicians and present model.

| | | Sensitivity | Specificity | PPV | NPV | Accuracy |
|---|---|---|---|---|---|---|
| Set 1 | Physicians | 0.47 | 0.82 | 0.55 | 0.78 | 0.72 |
| | Present Model | 1 | 0.8 | 0.9 | 1 | 0.93 |
| Set 2 | Physicians | 0.23 | 0.64 | 0.26 | 0.6 | 0.5 |
| | Present Model | 0.9 | 0.75 | 0.9 | 0.75 | 0.86 |
| Overall | Physicians | 0.41 | 0.78 | 0.47 | 0.74 | 0.67 |
| | Present Model | 0.95 | 0.77 | 0.9 | 0.87 | 0.9 |

In some embodiments of the present application, Step (A) of acquiring a first data set of a medical institution further includes filtering the first data set to exclude data of non-target patients in the first data set, wherein the data of non-target patients comprise data of non-adults, data of people who suffer an out-of-hospital cardiac arrest, data of people who discharge against medical advice, data of people who leave without notice, data of people who withdraw registrations, data of people who are transferred to other institutions, and data of people with incomplete data.

Screening the first data set and excluding the data of non-target patients can help improve the data quality and the study's effectiveness. Screening out the data of non-target patients (such as minors, out-of-hospital cardiac arrest patients, etc.) can ensure that the data set is more unified, thereby reducing the deviation caused by the significant difference in patient types. There may be large differences between minors and adults in pathological characteristics, treatment methods, etc. Screening out the data of minors can help make the research on the adult population more accurate. Some patients (such as those who abandon medical advice, leave without notice, cancel registration, or have incomplete data) may not complete the treatment process, resulting in incomplete or non-standard data. Excluding these data can avoid the impact of incomplete or abnormal data on the research results and improve the scientific and reliability of the conclusions. The treatment process of transferred patients and out-of-hospital cardiac arrest patients may affect the efficacy data due to external intervention. Screening out the data of such patients can ensure the sample's representativeness so that the medical conditions of the patients in the data set are consistent with the research objectives, which is convenient for subsequent analysis. Excluding the data of patients who do not meet the conditions can reduce data processing complexity and interference with the model training and analysis process, thereby improving the analysis efficiency. Models and analysis algorithms can focus more on qualified data and improve the stability of data processing and results. In addition, patients with incomplete data and abnormal treatment processes can quickly introduce bias, causing the model to make false associations or inaccurate predictions. Excluding these data helps reduce the negative impact of noise and outliers on the analysis, ensuring that the model and analysis results are more interpretable.

In some embodiments of the present application, Step (B) of preprocessing the first data set into a second data set further includes Step (B1) of grouping the first structured data based on a preset index and normalizing the first structured data, Step (B2) of optimizing the first unstructured data so that the first unstructured data matches rules of the medical model, wherein an optimization comprises at least one step of data cleaning, text translation, or text correction; wherein the medical model processes the first unstructured data using a natural language processing (NLP) algorithm, and the natural language processing algorithm is designed to process monolingual information. For example, reference quantities for grouping comprise age, mean arterial pressure (MAP), or body temperature; the step of grouping the first structured data based on a preset index and normalizing the first structured data, further comprising: performing a one-hot encoding on a grouped first structured data; normalizing a respiratory rate, a pulse rate and a blood oxygen saturation in the first structured data by a min-max normalization algorithm.

For example, patients are divided into 3 groups based on age: young (18 to 40 years old), middle-aged (41 to 64 years old), and elderly (greater than or equal to 65 years old). Mean arterial pressure is calculated to distinguish patients with shock from other patients, and patients are divided into fever and non-fever groups with a cutoff value of 38 degrees Celsius. Each category is encoded using one-hot encoding, and the minimum-maximum normalization algorithm is used to standardize the numerical data of respiratory rate, pulse, and blood oxygen saturation.

By dividing patients into young, middle-aged, and elderly groups according to age, the physiological differences of patients of different age groups can be analyzed more accurately, thereby capturing the characteristics of different age groups. For example, the mean arterial pressure and cardiovascular status of the elderly and young people are usually different. Such grouping helps to identify risk factors for different age groups. Mean arterial pressure can directly reflect whether the blood circulation is normal. It is used to distinguish shock patients from other patients, which helps to identify and mark potentially dangerous patient groups quickly. Through such a distinction, the model can focus more accurately on the characteristics of shock patients, thereby improving this group's predictive ability and judgment accuracy. The fever and non-fever groups are divided with 38 degrees Celsius as the critical value so that the model can identify the impact of body temperature on the condition. Fever may be related to infection or inflammation, so this grouping method helps to highlight the impact of fever on the patient's condition and supports more targeted analysis.

Using one-hot encoding for age groups, shock states, and fever states can convert categorical features into numerical features, making the model easier to process. One-hot encoding can prevent the model from misunderstanding the order relationship when processing categorical data and ensure that the impact of each categorical feature on the model is independent, thereby improving the model's performance. Using min-max normalization to scale the numerical data of respiratory rate, pulse, and blood oxygen saturation to the same range can reduce the impact of dimensional differences and increase the convergence speed of the model, thereby reducing the weight impact on model training caused by a significant feature value. In addition, normalization can also reduce the impact of outliers, making the model more stable and robust.

In some embodiments of the present application, data cleaning involves converting texts in the first unstructured data in abbreviated form into terms in fully expanded form. The data cleaning further includes retaining preset punctuation marks in the first unstructured data, deleting stop words in the first unstructured data, and punctuation marks other than the preset punctuation marks, wherein the preset punctuation marks are commonly used punctuation marks in the medical field. The data cleaning further includes retaining negative words in the first unstructured data; the text translation includes translating non-English texts in the first unstructured data into English texts. The text correction includes correcting spelling errors in the first unstructured data. The preset punctuation marks include a slash (“/”), a plus sign (“+”), a minus sign (“-”), and a space character (”).

Since the first unstructured data is free text data, it is difficult to analyze, and different doctors have different writing styles, such as abbreviations, punctuation, uppercase preference, etc. In addition, free text may include incomplete and ungrammatical sentences and Chinese characters; this is because Chinese is the official working language in Taiwan and Mandarin is widely used (the source of the data set in this application is in default from the Department of Health and Welfare of Taipei Medical University Shuanghe Hospital mentioned above). The above factors complicate text analysis because most existing natural language processing algorithms are designed to process monolingual information. If an additional natural language processing algorithm adapted to multiple languages is to be designed, the development cost will be significantly increased. And multilingual text will lead to poor prediction performance of the model because the model can only follow the grammatical principles of one language. Therefore, the first data set is preprocessed to solve the above problems. The following example is an excerpt of two original cases in the data set. Case 1: “Present Illness: VISIED ED 3 DAYS AGO, NO FEVER, NO ¬¬¬ CONTUSION, NO ABDOMINAL PAIN, TODAY, LEFT INGUINAL CARBUNCLE SINCE LAST WEEK WITH DISCHARGE”. Case 2: “Chief Complaint: ABD op hx: nil, deined CHRONIC dz, nkda, NO COLD SWEATING, NO RADIATION TO BACK, NO diarrhea, CT in China: ureter stone, rlq pain on and off, feel pain when press it, then ruq pain, chest XR, In China, feel chest is stuck after a meal, similar episode? gerd location, no radiation pain to back, persistent, acute onset, chest pain the date before yesterday”.

Because of these diverse language conditions, the following text refinement must be done: text translation, abbreviation expansion, spelling error correction, adjustment of uppercase letters to lowercase letters, and stopword and punctuation mark removal. For example, the Google Cloud Platform Translate application programming interface can translate Chinese characters into English words. Then, abbreviations or inappropriate sentences are converted to standard terms, such as converting “ABD” to “abdomen,” “OP” to “operation,” “Hx” to “history,” “NKDA” to “no known drug allergy,” “No pull” to “no diarrhea,” “RLQ” to “right lower quadrant,” etc. Correct spelling errors (such as converting “Parkenson” to “Parkinson's Disease”) and adjust some capital letters to lowercase (such as converting “NO RADIATION TO BACK” to “no radiation to back”). In addition, A dictionary of standard clinical terms compiled by emergency physicians is used to map abbreviations in the narrative text to the fully expanded terms. For example, the NLTK (natural language toolkit) package is used to remove stopwords and punctuation marks, except for the slash (“/”), plus sign (“+”), minus sign (“−”), and null character (“ ”) that are commonly used in medical abbreviations. For example, laceration is abbreviated as L/W, and abrasion is abbreviated as A/W. Some doctors prefer “+” or “−” to indicate whether the patient has a specific symptom. For example, dizziness (+) means that the patient is dizzy. Negative words in the texts are kept for stopword removal since negative findings are important for clinical judgment. For example, in an original text, “no fever, no SOB” would be converted to “fever, SOB” if “no” is removed, which has an entirely different meaning.

In some embodiments of the present application, the second structured data includes vital signs and demographic data, and the second unstructured data includes a chief complaint, a present illness, and a medical history; the first type of data is related to the vital signs and the demographic data, and a dimension of the first type of data is 15, and dimensions of the data related to the chief complaint and the medical history in the second type of data and the third type of data are between 100-500. The second type of data includes a first sub-data and a second sub-data. The dimensions of the first sub-data and the second sub-data are both 300. The third data type includes a third sub-data, a fourth sub-data, and a fifth sub-data. The dimensions of the third sub-data and the fifth sub-data are both 200, and the dimension of the fourth sub-data is 30, wherein the first sub-data is obtained by processing the chief complaint through the bidirectional long short-term memory network, the second sub-data is obtained by processing the present illness through the bidirectional long short-term memory network, the third sub-data is obtained by extracting linguistic features from the chief complaint, the fourth sub-data is obtained by extracting linguistic features from the medical history, and the fifth sub-data is obtained by extracting linguistic features from the present illness. The step of extracting linguistic features from the second unstructured data to obtain a third type of data, further comprising: when the second unstructured data contains keywords, extract the keywords from the second unstructured data, and obtain the third type of data based on the keywords and corresponding weights; when the second unstructured data do not contain keywords, average values of all word vectors in the second unstructured data are calculated, and cosine similarities between the average values and all preset keyword vectors is calculated, and five preset keyword vectors with the closest distance are selected as the third type of data.

FIG. 2 shows a schematic diagram of a medical model provided in an embodiment of the present application. There are some abbreviations in FIG. 2, and their meanings are as follows: CNTR means clinical narratives text representation, BILSTM means bidirectional long short-term memory network, kw means keyword, and dim means dimension.

In this application, different processing methods are used for different input data. For the chief complaint and present illness, the dimensions of these two data types can be selected between 100-500. 100, 200, 300, 400, and 500 are selected as the target dimensions for experiments, and the following conclusions are drawn: for the chief complaint (corresponding to the first sub-data above) and the present illness (corresponding to the second sub-data above) obtained by bidirectional long short-term memory network processing, their dimensions are set to 300; for the chief complaint (corresponding to the third sub-data above) and the present illness (corresponding to the fifth sub-data above) obtained by language feature extraction, their dimensions are set to 200 (the dimensions of positive vectors and negative vectors are 100 respectively). The dimension of the medical history (corresponding to the fourth sub-data above) is set to 30, and the dimension of the processed vital signs and demographic data (corresponding to the first type of data above) is set to 15. In this way, the use effect of the model can be optimized. The processing method for the first data type includes the one-hot encoding and min-max normalization algorithm disclosed above.

Clinical narrative-aware deep neural networks predict critical outcomes in the pre-diagnosis stage by processing text-intensive data and standardized numerical data. Referring to FIG. 2, the medical model disclosed in this application consists of five main parts: preprocessing, language feature extraction, bidirectional long short-term memory network, a multi-feature fusion mechanism, and critical outcome prediction.

Since the performance of text classification benefits from efficient text representation, we present the Clinical Narratives Text Representation (CNTR) based on embeddings, which is used in this study to develop a correlation between critical outcomes and clinical narratives (i.e., medical history, chief complaints, and present illness). The inspiration behind CNTR comes from the observation of how critical care physicians can identify an ICU case by means of critical lexicon or semantic contents to narrow the scope of possible candidates rapidly. For instance, when an expression contains keywords such as “ICH (intracerebral hemorrhage),” “SAH (subarachnoid hemorrhage),” and “aortic dissection,” we can conclude that the expression very likely contains crucial information. This assumption can be used to explain how critical care physicians can browse through the clinical narrative quickly to capture key information about the ICU case. The CNTR method first screens out keywords related to critically ill patients (IHCA patients/ICU patients) from all categories of clinical texts and combines them into a keyword dictionary. Based on the keyword dictionary, it is determined whether the clinical text contains keywords. If so, the text is represented by the log-likelihood ratio (LLR) weight. When the clinical text does not contain any keywords, it is represented by a weighted combination of several keywords with the highest similarity to the entire clinical text, where the similarity score between the keyword and the text determines the weight.

This application uses a category-based keyword extraction method that calculates term weights based on the association between terms and categories. In this way, high-weight terms represent a strong association with ED critical outcomes. This association is determined by calculating the log-likelihood ratio of each word in the text. Patients with a high risk of admission to the ICU or IHCA are defined as positive cases, and patients with a low risk are defined as negative cases. The calculation formula is as follows:

$$LLR ( w , HR ) = 2 log [ \frac{{p ( w \vert HR )}^{k} {( 1 - p ( w \vert HR ) )}^{m} {p ( w \vert ¬ HR )}^{l} {( 1 - p ( w \vert ¬ HR ) )}^{n}}{{p ( w )}^{k + l} {( 1 - p ( w ) )}^{m + n}} ] ;$$

HR represents a high-risk patient (high risk of admission to ICU or IHCA), and ¬ HR represents a non-high-risk patient. w represents a high-risk keyword, k represents the number of clinical narratives of high-risk cases containing w, l represents the number of clinical narratives containing w but not high-risk cases, m represents the number of clinical narratives of high-risk cases without w, and n represents the number of clinical narratives of non-high-risk cases without w. The higher the LLR value, the stronger the relationship between the word and a certain ED disposition. Maximum likelihood estimation is implemented to obtain the following probability values: p(w), p(w|HR), and p(w|¬HR). This formula is used to measure the difference between the keyword w and whether the patient has a high-risk (HR) situation, and this method is applied to clinical texts to extract key information in clinical texts.

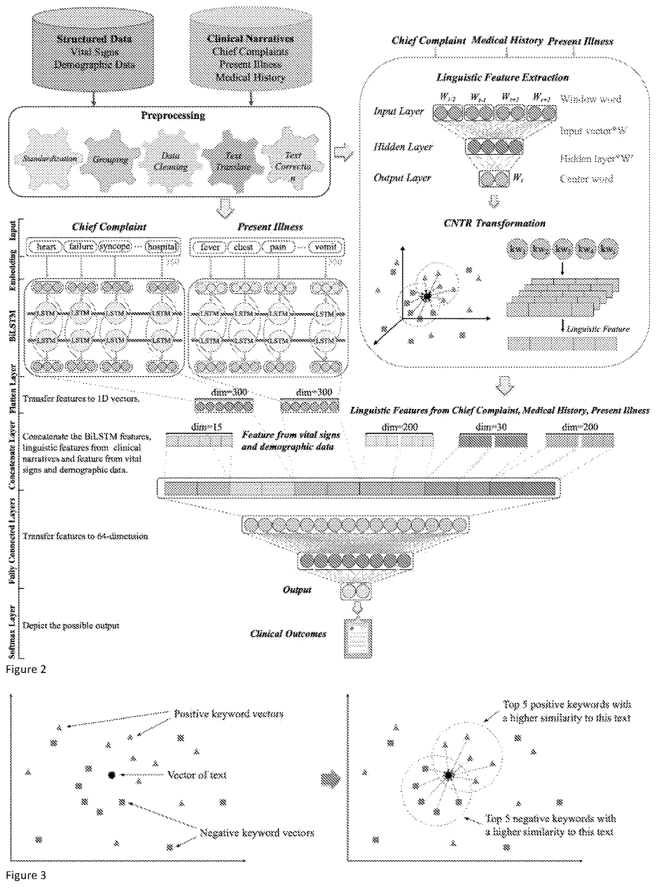

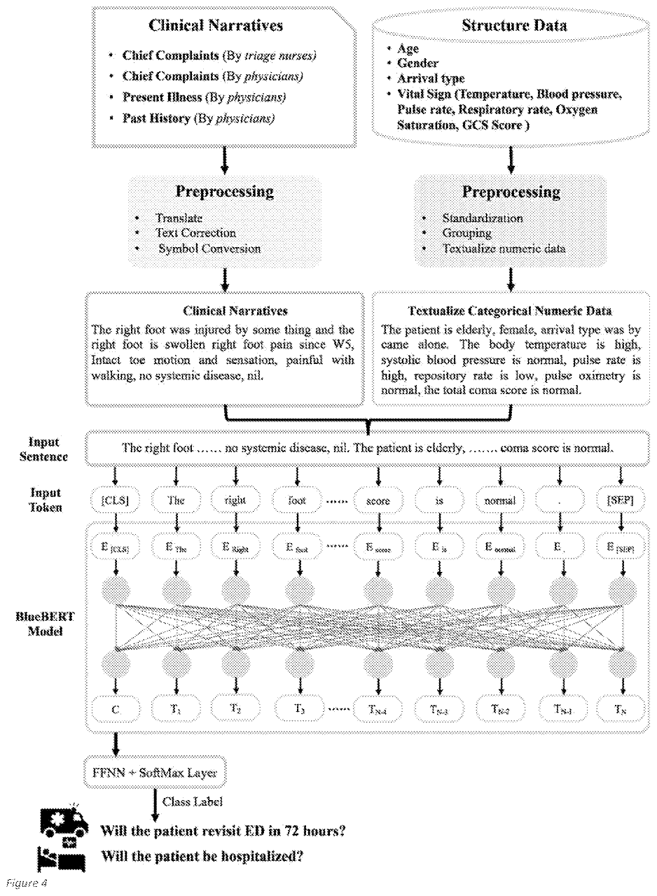

After this, LLR values of all words are sorted in the training data, and the top 200 candidates are kept as a keyword list for both chief complaints and present illness. Since the content in medical history is listed as an item description composed of a couple of keywords, the top 30 are therefore selected as the keyword list for medical histories. Next, Gensim is employed to train word embeddings for text representation with the continuous Bag-of-Words (CBOW) method. In this application, word embeddings are trained using the clinical narrative of 171,275 ED visits, including 11,060,526 tokens, in which 299,591 unique words are encoded as 300-dimensional distributed vectors. The keyword embeddings jointly represent the clinical narratives. More specifically, the clinical narrative text Tₖ is represented as a weighted average of the keyword vectors, and the weight λᵢ for a keyword Kᵢ is determined by its LLR value. FIG. 3 shows a method for processing clinical narratives without any keyword provided in an embodiment of the present application. In FIG. 3, for a clinical narrative without any keywords, the average value of all word vectors in the clinical narrative is calculated, and the cosine similarity of all keyword vectors is calculated to find the keywords closest to representing the clinical narrative. For example, the number of selected keywords is 5. In FIG. 3, the similarity between the text and each keyword vector can be intuitively determined. The closer the distance between them, the higher the similarity; conversely, the farther the distance between them, the lower the similarity.

In some embodiments of the present application, a 10-fold cross-validation method can be used for validation. For example, the data set is divided into ten (10) equal parts, nine (9) of which are used to train the model, and one (1) is used for validation. This helps to evaluate the generalization ability of the CNTR model on clinical narrative text data and ensures that the model performs stably on unseen data. When processing medical text, the data may be small or uneven, and cross-validation can effectively reduce the impact of the randomness of data segmentation on model evaluation. During validation, the dropout parameter is set to 0.35. Dropout is a technique to prevent model overfitting by randomly discarding a portion of neurons (35% in this application) during training to reduce the model's dependence on specific neurons. In CNTR, text data is usually high-dimensional and sparse. The model can be more robust through appropriate dropout when processing clinical text (such as doctor's notes and medical records). The number of training epochs is set to 40. The number of training epochs determines the length of time the model learns the data. Forty (40) cycles mean the model will perform 40 complete training rounds on the clinical narrative text. If the number of cycles is too low, the model may not capture enough semantic information; if too high, it may lead to overfitting. In this application, experiments show that the data features can be effectively learned when the number of training cycles is 40. The threshold is set to 0.5. Thresholds are usually used for classification tasks, especially in binary or multi-classification problems. 0.5 means that the predicted value is considered a positive class when greater than or equal to 0.5, and vice versa for negative classes. In CNTR, the model may need to classify the patient's state or condition based on the input text content (such as descriptions related to “shock”), and the threshold setting directly affects the judgment criteria of the classification results. An early stopping strategy can be used to prevent the model from overfitting. If the performance of the validation set does not improve within three (3) consecutive cycles, the training is stopped. In CNTR, overfitting causes the model to over-memorize the training data and reduce the generalization ability of new text. Early stopping helps optimize training efficiency and prevent performance degradation. Set the maximum length (max_length) of each text to 350. When processing text, a max_length of 350 means that each clinical text contains a maximum of 350 units, with the excess truncated and the insufficient padded. The length of medical texts may vary greatly. By unifying the length, the model can process text data more efficiently while avoiding the computational overhead caused by overly long texts. In addition, data processing can be performed using synthetic minority oversampling technology (SMOTE). When CNTR processes clinical texts, it may face the problem of imbalanced data categories, such as less data for a particular type of disease description and more data for other categories. SMOTE balances the data distribution by synthesizing minority category samples. The generated 1:1 balanced ratio allows the model to give equal attention to each data category, avoid the model's bias towards multi-category data, and improve the prediction performance on minority categories (such as rare disease descriptions).

In addition to the linguistic feature extraction by the CNTR, the text of the chief complaints and present illness are inputted into the deep neural network. The embedding layer is first used to transform the input tokens of a sentence into 300-dimension vectors. Furthermore, the maximum document length is set to 350, where longer documents are truncated, and shorter documents are padded with zeros. Next, the latent semantic features behind the text of the chief complaints and present illness are learned through two (i.e., bichannel) BILSTM, which can consider past and future data information by connecting two LSTM networks with opposite timings in the same output. Deep learning can unearth the latent features by itself, and this usually relies on language input instead of feature engineering. However, many feature vectors can be put together with the neural networks to enhance the performance. In this application, the generated CNTRs of clinical narratives are integrated into the neural network by concatenating positive and negative vectors, i.e., a 30-dimension vector for medical history and two 200-dimension vectors for chief complaints and present illness, respectively. As a result, the merging of these features into the deep learning model is discriminative.

In another specific embodiment, the above medical model of the present invention is designed to determine whether a patient visiting the emergency room will actually be hospitalized. More specifically, the medical model of the present invention may assist in determining (1) whether a patient will be hospitalized and (2) whether the patient will return to the emergency room within 72 hours of discharge. This study used the electronic health records from the Emergency Department of Taipei Medical University-Shuang Ho Hospital, Ministry of Health and Welfare, for model development and consisted of a total of 245,721 emergency visits.

The medical model building method used in the present embodiment is similar to the previous one and is, therefore, described only regarding the differences. Referring to FIG. 4, during the pre-processing stage of this medical model building method, the first structured data and the first unstructured data in the first dataset are pre-processed into the second structured data and the second unstructured data, respectively. Importantly, the text and numbers are pre-processed separately in the pre-processing stage, and the text is further translated into English if the first structured data and the first unstructured data are presented in a non-English text forms). Therefore, the above contents are Steps S100 and S200.

In a specific embodiment of the present invention, the above medical model is constructed to assess whether the patient visiting an emergency clinic is truly in critical condition. After the medical model is built, its performance is tested. As Table 3 shows, AUROC and AUPRC of Disposition are 0.9014 and 0.7739, better than other related models in the prior arts. Further, AUROC and AUPRC of Unscheduled Return Visits are 0.6475 and 0.0539, which are also better than other related models in the prior arts. In addition, the present medical model also shows that the method including Translated Text and Textualize Categorical Numeric Data has the highest AUROC and AUPRC values (as Table 4 shows).

TABLE 3
Performance of compared models

| Experiment | AUROC | AUPRC | Sensitivity | Specificity | PPV | NPV |
|---|---|---|---|---|---|---|
| | Disposition | | | | | |
| CNB | 0.8159 | 0.6284 | 0.3464 | 0.9639 | 0.7495 | 0.8254 |
| KNN | 0.7721 | 0.5935 | 0.3857 | 0.3991 | 0.6641 | 0.8305 |
| RF | 0.8494 | 0.6743 | 0.3084 | 0.9778 | 0.8125 | 0.8193 |
| XGB | 0.8521 | 0.6905 | 0.4280 | 0.9590 | 0.7651 | 0.8431 |
| LGBM | 0.8569 | 0.6970 | 0.4412 | 0.9582 | 0.7671 | 0.8461 |
| BioClinicalBERT | 0.8797 | 0.7345 | 0.6211 | 0.9201 | 0.7085 | 0.8862 |
| BlueBERT | 0.8798 | 0.7349 | 0.6238 | 0.9208 | 0.7106 | 0.8870 |
| Present Model | 0.9014 | 0.7739 | 0.8037 | 0.8608 | 0.6760 | 0.9184 |
| | Unscheduled Return Visits | | | | | |
| CNB | 0.5895 | 0.0516 | 0.0856 | 0.9804 | 0.0909 | 0.9791 |
| KNN | 0.5763 | 0.0518 | 0.1929 | 0.9018 | 0.0432 | 0.9799 |
| RF | 0.6091 | 0.0490 | 0.0876 | 0.9798 | 0.0906 | 0.9791 |
| XGB | 0.5923 | 0.0440 | 0.1178 | 0.9570 | 0.0592 | 0.9793 |
| LGBM | 0.6104 | 0.0516 | 0.1122 | 0.9642 | 0.0670 | 0.9793 |
| BioClinicalBERT | 0.6262 | 0.0498 | 0.1905 | 0.9181 | 0.0523 | 0.9802 |
| BlueBERT | 0.6334 | 0.0510 | 0.2172 | 0.9085 | 0.0522 | 0.9806 |
| Present Model | 0.6475 | 0.0539 | 0.2435 | 0.8991 | 0.0532 | 0.9811 |

TABLE 4
Performance of compared methods

| Experiment | AUROC | AUPRC | Sensitivity | Specificity | PPV | NPV |
|---|---|---|---|---|---|---|
| | Disposition | | | | | |
| Baseline | 0.8798 | 0.7349 | 0.6238 | 0.9208 | 0.7106 | 0.8870 |
| +TT | 0.8891 | 0.7513 | 0.7948 | 0.8561 | 0.6508 | 0.9201 |
| +TT + ND | 0.7922 | 0.7303 | 0.7987 | 0.8574 | 0.6679 | 0.9164 |
| +TT + CND | 0.7873 | 0.7283 | 0.7966 | 0.8574 | 0.6536 | 0.9210 |
| +TT + CNDT | 0.9014 | 0.7739 | 0.8037 | 0.8608 | 0.6760 | 0.9184 |
| | Unscheduled Return Visits | | | | | |
| Baseline | 0.6334 | 0.0510 | 0.2172 | 0.9085 | 0.0522 | 0.9806 |
| +TT | 0.6455 | 0.0561 | 0.2092 | 0.9192 | 0.0567 | 0.9807 |
| +TT + ND | 0.5696 | 0.1554 | 0.2421 | 0.8971 | 0.0518 | 0.9810 |
| +TT + CND | 0.5668 | 0.1488 | 0.2262 | 0.9074 | 0.0540 | 0.9808 |
| +TT + CNDT | 0.6475 | 0.0539 | 0.2435 | 0.8991 | 0.0532 | 0.9811 |

Note:
Baseline = Non-Translated Text,
TT = Translated Text,
ND = Nmneric Data,
CND = Categorical Numeric Data,
CNDT = Textualize Categorical Numeric Data

1. A method for constructing a medical model, wherein the medical model is configured to predict a diagnosis result based on a subject's information, comprising:
(A) acquiring a first data set of a medical institution, wherein the first data set comprises a first structured data and a first unstructured data;
(B) preprocessing the first data set into a second data set, wherein the second data set comprises a second structured data and a second unstructured data;
(C) obtaining a first type of data based on the second structured data, and processing the second unstructured data by a bidirectional long short-term memory network to obtain a second type of data, and extracting linguistic features from the second unstructured data to obtain a third type of data; and
(D) fusing the first type of data, the second type of data, and the third type of data to get a fused result, wherein the fused result is processed using a softmax function to obtain the diagnosis result.

2. The method according to claim 1, wherein Step (B) further comprises:
(B1) grouping the first structured data based on a preset index and normalizing the first structured data;
(B2) optimizing the first unstructured data so that the first unstructured data matches a rule of the medical model, wherein an optimization comprises at least one step of data cleaning, text translation, or text correction,
wherein the medical model processes the first unstructured data using a natural language processing algorithm, and the natural language processing algorithm is designed to process monolingual information.

3. The method according to claim 2, wherein a reference quantity for grouping comprises age, mean arterial pressure, or body temperature.

4. The method according to claim 2, wherein Step (B1) comprises:
performing a one-hot encoding on a grouped first structured data;
normalizing a respiratory rate, a pulse rate, and a blood oxygen saturation in the first structured data by a min-max normalization algorithm.

5. The method according to claim 2, wherein the data cleaning comprises converting texts in the first unstructured data in abbreviated form into terms in fully expanded form.

6. The method according to claim 2, wherein the data cleaning comprises retaining preset punctuation marks in the first unstructured data and deleting stop words in the first unstructured data and punctuation marks other than the preset punctuation marks, wherein the preset punctuation marks are commonly used punctuation marks in a medical field.

7. The method according to claim 6, wherein the data cleaning comprises retaining negative words in the first unstructured data.

8. The method according to claim 6, wherein the preset punctuation marks comprise: a slash, a plus sign, a minus sign, and a space character.

9. The method according to claim 2, wherein the text translation comprises translating non-English texts in the first unstructured data into English texts.

10. The method according to claim 2, wherein the text correction comprises correcting spelling errors in the first unstructured data.

11. The method according to claim 1, wherein Step (A) further comprises: filtering the first data set to exclude data of non-target patients in the first data set, wherein the data of non-target patients comprise data of non-adults, data of people who suffer an out-of-hospital cardiac arrest, data of people who discharge against medical advice, data of people who leave without notice, data of people who withdraw registrations, data of people who are transferred to other institutions, and data of people with incomplete data.

12. The method according to claim 1, wherein the diagnosis result comprises a critical outcome or a non-critical outcome, wherein the critical outcome comprises an in-hospital cardiac arrest or an admission to an intensive care unit, and the non-critical outcome comprises a discharge or an admission to a ward.

13. The method according to claim 1, wherein the second structured data comprises vital signs and demographic data, and the second unstructured data comprises a chief complaint, a present illness, and a medical history; and
the second type of data comprises a first sub-data and a second sub-data, and the third type of data comprises a third sub-data, a fourth sub-data, and a fifth sub-data,
wherein the first sub-data is obtained by processing the chief complaint through the bidirectional long short-term memory network, the second sub-data is obtained by processing the present illness through the bidirectional long short-term memory network, the third sub-data is obtained by extracting linguistic features from the chief complaint, the fourth sub-data is obtained by extracting linguistic features from the medical history, and the fifth sub-data is obtained by extracting linguistic features from the present illness.

14. The method according to claim 1, wherein the first sub-data is related to the vital signs and the demographic data.

15. The method according to claim 13, wherein a dimension of the data related to the chief complaint and the medical history in the second type of data and the third type of data is between 100-500.

16. The method according to claim 13, wherein a dimension of the first sub-data and the second sub-data are both 300.

17. The method according to claim 13, wherein dimensions of the third sub-data and the fifth sub-data are both 200.

18. The method according to claim 13, wherein a dimension of the fourth sub-data is 30.

19. The method according to claim 1, wherein a dimension of the first type of data is 15.

20. The method according to claim 1, wherein a dimension of the fused result is 64.

21. The method according to claim 1, wherein the step of fusing the first type of data, the second type of data and the third type of data to get a fused result comprises following calculation formulas:
$$F = DF ⊕ VF ⊕ LF ⊕ LSF$$
$$\hat{y} = softmax ( {MF}^{T} + b )$$
wherein F is a result of the fusion, DF is a feature related to the demographic data in the first type of data, VF is a feature related to the vital signs in the first type of data, LF is the third type of data, LSF is the second type of data, ŷ is an approximate probability value, M is a parameter matrix of a connection layer, and b is a bias.

22. The method according to claim 1, wherein the step of “extracting linguistic features from the second unstructured data to obtain a third type of data” further comprises:
when the second unstructured data contain keywords, extract the keywords from the second unstructured data, and obtain the third type of data based on the keywords and corresponding weights; and
when the second unstructured data do not contain keywords, average values of all word vectors in the second unstructured data are calculated, and cosine similarities between the average values and all preset keyword vectors is calculated, and five preset keyword vectors with a closest distance are selected as the third type of data.

23. The method according to claim 22, wherein a weight calculation formula is as follows:
$$LLR ( w , HR ) = 2 log [ \frac{{p ( w \vert HR )}^{k} {( 1 - p ( w \vert HR ) )}^{m} {p ( w \vert ¬ HR )}^{l} {( 1 - p ( w \vert ¬ HR ) )}^{n}}{{p ( w )}^{k + l} {( 1 - p ( w ) )}^{m + n}} ]$$
wherein HR is a high-risk patient, ¬ HR is a non-high-risk patient, w represents a keyword of high risk, k represents a quantity of clinical narratives of high-risk cases including w, l represents a quantity of clinical narratives including w but not high-risk cases, m represents a quantity of clinical narratives of high-risk cases without w, and n represents a quantity of clinical narratives of non-high-risk cases without w.

24. The method according to claim 1, wherein the medical model is constructed using following parameters:
a validation method is 10-fold cross-validation, a dropout is 0.35, training epochs are 40, a threshold is 0.5, a parameter of the early stopping strategy is 3, and a maximum length of each text is 350; and
the method further comprises:
a synthetic minority class oversampling technique is used to balance a data distribution so that a proportion of data in each class is equal.

25. A medical system, comprising:
a medical model, which is constructed using the method as described in claim 1, wherein the medical model is configured to predict a diagnosis result based on information of target patients.

26. A non-transitory computer-readable storage medium having computer-executable instructions stored thereon, which when executed by one or more processors, cause the one or more processors to perform operations comprising the method according to claim 1 for constructing a medical model to predict a diagnosis result based on a subject's information.
